(19) United States
(12) Patent Application Publication
(10) Pub. No.: US 2014/0335132 A1
(43) Pub. Date: Nov. 13, 2014

(54) BINDING DRUGS WITH NANOCRYSTALLINE CELLULOSE (NCC)

(71) Applicants: Helen Mary Burt, Vancouver (CA); John Kevin Jackson, Vancouver (CA); Wadood Yasser Hamad, Vancouver (CA)

(72) Inventors: Helen Mary Burt, Vancouver (CA); John Kevin Jackson, Vancouver (CA); Wadood Yasser Hamad, Vancouver (CA)

(21) Appl. No.: 13/885,503

(22) PCT Filed: Nov. 22, 2011
(86) PCT No.: PCT/CA2011/001281

§ 371 Date: Jul. 18, 2013

Related U.S. Application Data
(60) Provisional application No. 61/416,494, filed on Nov. 23, 2010.

(51) Int. Cl. A61K 47/48; A61K 31/65; A61K 45/06; A61K 31/704

(52) U.S. Cl. CPC A61K 47/4823; A61K 31/704; A61K 31/65; A61K 45/06
USPC 424/400; 536/6.4; 536/56; 514/34; 514/57; 525/54.21

(57) ABSTRACT

This invention describes nanocrystalline cellulose (NCC) for use as a drug delivery excipient. NCC binds significant quantities of water soluble, ionizable drugs, e.g., tetratcycline and doxorubicin, which are released rapidly over a one day period. A surfactant such as cetyl trimethylammonium bromide (CTAB) can bind to the surface of NCC and increase the zeta potential in a concentration-dependent manner from −55 to 0 mV. NCC with CTAB modified surfaces can bind significant quantities of the hydrophobic drugs such as anticancer drugs docetaxel, paclitaxel and etoposide. These drugs were released in a controlled manner over a 2-day period. The NCC-CTAB nanocomplexes were found to bind to KU-7 cells and evidence of cellular uptake was observed.

### TECHNICAL FIELD

This invention relates to nanocrystalline cellulose (NCC) for use in the binding and release of drugs including a range of ionized drugs, and the use of surface modified NCC, e.g. with the surfactant cetyl trimethylammonium bromide (CTAB), for the binding and release of hydrophobic drugs. The invention also relates to a pharmaceutical composition comprising a drug bound to NCC; to a process for producing such a pharmaceutical composition; and to a method of treatment with such a pharmaceutical composition.

### BACKGROUND ART

Cellulose has a long history of use in the pharmaceutical industry. The material has excellent compaction properties when blended with other pharmaceutical excipients so that drug-loaded tablets form dense pellets suitable for swallowing and the oral administration of drugs. The form of cellulose used in tablets is termed microcrystalline cellulose (MCC) which is a purified, depolymerised alpha cellulose derived from plant sources [1]. Despite an extended history of use in tableting, there is still considerable continuing research into the use of MCC and other types of cellulose in advanced pelleting systems whereby the rate of tablet disintegration and drug release may be controlled by microparticle inclusion, excipient layering or tablet coating [2-11].

Derivatized cellulose has also been used extensively in pharmaceutical preparations so that ethyl cellulose, methyl cellulose, carboxymethyl cellulose and numerous other forms are used in both oral, topical and injectable formulations. For example carboxymethyl cellulose is the primary component of “Seprafilm™” which is applied to surgical sites to prevent post surgical adhesions. Recently, the use of MCC in self emulsifying drug delivery systems and semi solid injectable formulations has been described [2, 12]. The use of these forms of cellulose in such formulations points to the inertness and excellent biocompatibility of cellulose in humans. Indeed, hydroxypropyl methyl cellulose has recently been used as a hydrogel matrix for chondrocyte implantation into animal joints for cartilage repair [13].

However, these uses of cellulose in formulations do not involve direct molecular level control of drug release via binding interactions with the drug. Although the surface of MCC has a slight negative charge due to hydroxyl residues, this charge is confined to a relatively small surface area on a large mass of insoluble MCC and would not likely adsorb or bind significant amounts of drug. The principle of using charged particles to bind acidic or basic drugs is well established since ion-exchange resins have been used for 50 years to bind and release drugs [14]. Drug release from resins is usually rapid once the resin-drug complex reaches the target site since counter ions present in body fluids displace the drug from the binding site. For extended release, resins have been coated with agents such as ethyl or carboxymethyl cellulose to delay drug elution. Complex polysaccharides such as chitosan have been used extensively in controlled release drug formulations. Such methods rely on a charge interaction between the positive charge on amine groups of each sugar residue in chitosan with negatively charged drugs such as antisense oligonucleotides [15, 16]. The high positive and negative charges on chitosan and oligonucleotides respectively, allow for a strong binding interaction between the carrier and the drug so that phosphate counterions tend to release a weakly-bound fraction rapidly and a tightly-bound fraction very slowly [16].

Nanocrystalline cellulose (NCC) is extracted from woody or non-woody biomass (e.g. bleached kraft wood pulp) using an acid hydrolytic extraction process. NCC has a very high surface area to volume ratio due to the nanometer size of the NCC crystals. Other nanocrystalline material, such as nanocrystalline clays have been shown to bind drugs and release them in a controlled manner via ion exchange mechanisms and are being investigated for use in pharmaceutical formulations [17]. The excellent established biocompatibility of cellulose supports the use of this material for similar purposes.

The very large surface area and negative charge of NCC suggests that large amounts of drugs might be bound to the surface of this material with the potential for high payloads and optimal control of dosing. Although un-ionized and/or hydrophobic drugs would not normally bind to such materials, other workers have suggested modification of charged surfaces with hydrophobic moieties to facilitate adsorption. Lonnberg et al., [18] suggested that poly(caprolactone) chains might be conjugated onto nanocrystalline cellulose for that purpose. However, there are no reports of the successful use of these methods to bind drugs to the NCC surface and subsequently release them in a controlled manner.

### DISCLOSURE OF THE INVENTION

This invention seeks to provide a pharmaceutical composition comprising NCC as a carrier for a drug.

This invention also seeks to provide a process for producing a pharmaceutical composition comprising NCC as a carrier for a drug.

Furthermore this invention seeks to provide a method of medical treatment in which NCC is a carrier for a drug.

Still further this invention seeks to provide the use of NCC as a carrier for a drug.

In accordance with one aspect of the invention there is provided a pharmaceutical composition comprising a drug bound to a carrier comprising nanocrystalline cellulose (NCC).

In accordance with one aspect of the invention there is provided a process of producing a pharmaceutical composition comprising binding a drug to a carrier comprising nanocrystalline cellulose (NCC).

In accordance with still another aspect of the invention there is provided in a method of treating or preventing a disease or ailment in which a drug is administered in a dosage form to a patient in need, the improvement wherein the drug is bound to a carrier comprising nanocrystalline cellulose (NCC).

In accordance with yet another aspect of the invention there is provided nanocrystalline cellulose (NCC) for use as a carrier for a drug in a pharmaceutical composition,

In accordance with a further aspect of the invention there is provided use of nanocrystalline cellulose (NCC) in the manufacture of a pharmaceutical composition in which the NCC is a carrier for a drug.

### BRIEF DESCRIPTION OF THE DRAWINGS

FIG. 1A illustrates graphically the binding of doxorubicin to 2 mg NCC in 10 mM, pH 7.4 PBS at 25° C.;

FIG. 1B illustrates graphically the binding of doxorubicin to 2 mg NCC in distilled water at 25° C.;

FIG. 2A illustrates graphically the binding of tetracycline to 2 mg NCC in 10 mM, pH 7.4 PBS at 25° C.;

FIG. 2B illustrates graphically the binding of tetracycline to 2 mg NCC in distilled water at 25° C.;

FIG. 3A illustrates graphically the binding of docetaxel to 2.5 mg of NCC/CTAB nanocomplexes in 10 mM NaCl at 25° C. with CTAB concentrations of 0 mM (▪), 0.755 mM (▴), 1.51 mM (▾), 2.27 mM (♦), 4.53 mM (), 6.79 mM (□), and 12.9 mM (A);

FIG. 3B illustrates graphically the maximal binding of docetaxel at a CTAB concentration of 12.9 mM;

FIG. 4A illustrates graphically the binding of paclitaxel to 2.5 mg of NCC/CTAB nanocomplexes in 10 mM NaCl at 25° C. with CTAB concentrations of 0 mM (▪), 0.755 mM (▴), 1.51 mM (▾), 2.27 mM (♦), 4.53 mM (), 6.79 mM (□), and 12.9 mM (A);

FIG. 4B illustrates graphically the maximal binding of paclitaxel at a CTAB concentration of 12.9 mM;

FIG. 5 illustrates graphically the binding of etoposide to 2.5 mg of NCC/CTAB nanocomplexes in 10 mM NaCl at 25° C. with CTAB concentrations of 0 mM (▪), 0.375 mM (▴), 0.755 mM (▾), 1.51 mM (♦), 2.27 mM (), 4.53 mM (□), and 6.79 mM (Δ) and 12.9 (∇);

FIG. 6 illustrates graphically the in vitro release of doxorubicin (Δ) and tetracycline (□) from NCC in 10 mM PBS at 37° C.;

FIG. 7 illustrates graphically the in vitro release of etoposide (∇), docetaxel (□) and paclitaxel (Δ) from NCC/CTAB nanocomplexes with 12.9 mM CTAB in 10 mM PBS at 37° C.;

FIG. 8 illustrates graphically the zeta potential of NCC/CTAB system as a function of CTAB concentration;

FIG. 9 illustrates graphically the mass of fluorescein bound to KU-7 cells as a function of concentration of fluorescein added to cells;

FIGS. 10A, B, C and D are confocal micrographs of KU-7 cells incubated for 2 hours with NCC/CTAB/fluoroscein system with a fluorescein concentration of 0.25 mg/ml. (A) White light image of KU-7 cells. (B) Staining of the nuclei with DAPI. (C) Fluorescein in the cytoplasm. (D) An overlay of images B and C.

### DETAILED DESCRIPTION OF THE INVENTION

This invention describes the potential use of nanocrystalline cellulose (NCC) as a drug delivery excipient for use alone or in conjunction with other formulations. In this invention it has been demonstrated that NCC is capable of binding significant quantities of ionizable water soluble antibiotics such as tetracycline and doxorubicin. These hydrophilic drugs were rapidly released with complete release in one day from formulations in which they were bound to NCC. By this invention it is also shown that the surface of NCC can be modified by binding a cationic surfactant, such as CTAB, resulting in a concentration-dependent increase in the zeta potential of the NCC crystallites. CTAB-coated NCC has further been shown to bind significant quantities of un-ionized hydrophobic therapeutic agents such as the anticancer agents docetaxel, paclitaxel and etoposide, and to release these drugs in a controlled manner over several days. The NCC/CTAB system also binds to KU-7 bladder cancer cells and has demonstrated efficient delivery of a hydrophobic fluorescent probe, fluoroscein, to the cytoplasm of these cells.

Nanocrystalline cellulose (NCC) herein refers to crystalline cellulose in which the crystals are of a particle size in the nano range, i.e. from 5 nm to 1000 nm. In this respect the particle size is the dimension corresponding to the diameter of a sphere encasing the nanoparticle.

Nanocrystalline cellulose (NCC) is extracted as a colloidal suspension by acid hydrolysis, especially with sulphuric acid, of cellulosic materials, such as bacteria, cotton, and wood pulp. NCC is constituted of cellulose, a linear polymer of β(1→4) linked D-glucose units, the chains of which arrange themselves to form crystalline and amorphous domains.

NCC obtained via hydrolytic extraction has a degree of polymerization (DP) in the range 90≦DP≦110, and 3.7-6.7 sulphate groups per 100 anhydroglucose units. NCC comprises crystallites whose physical dimension ranges between 5-10 nm in cross-section and 20-100 nm in length, depending on the raw material used in the extraction. These charged crystallites can be suspended in water, or other solvents if appropriately derivatized, or self assemble to form solid materials via air, spray- or freeze-drying. When dried, NCC forms an agglomeration of parallelepiped rod-like structures, which possess cross-sections in the nanometer range (5-20 nm), while their lengths are orders of magnitude larger (100-1000 nm) resulting in high aspect ratios. The iridescence of NCC self-assemblies is typically characterized by the finger-print patterns, where the patch work of bright and dark regions is typical of spherulitic behaviour of fibrillar crystals in which the molecules are packed with their axes perpendicular to the fibrillar axis. NCC is also characterized by high crystallinity (>80%, and most likely between 85 and 97%) approaching the theoretical limit of the cellulose chains.

Colloidal suspensions of cellulose crystallites form a chiral nematic structure upon reaching a critical concentration. The cholesteric structure consists of stacked planes of molecules aligned along a director (n), with the orientation of each director rotated about the perpendicular axis from one plane to the next. This structure forms spontaneously in solutions of rigid, rod-like molecules.

Hydrogen bonding between cellulose chains can stabilize the local structure in NCC, and plays a key role in the formation of crystalline domains. Crystallinity, defined as the crystalline fraction of the sample, strongly influences the physical and chemical behaviour of NCC. For example, the crystallinity of NCC directly influences the accessibility for chemical derivatization, swelling and water-binding properties.

The NCC functions as a carrier for the active drug of the pharmaceutical composition and additionally functions as a filler for the pharmaceutical composition in establishing a convenient and suitable dosage form for administration.

Since the drug and the NCC interact such that the drug is releaseably bound by the NCC, the NCC also functions to provide a controlled release of the drug on administration, for example a slow release or a release which is slower than that achieved by simple mixtures of drug and carrier or filler when there is no interaction.

The NCC may bear anionic charges which will bind an ionic drug, such anionic charges resulting from hydroxyl residues or from anionic acid groups such as sulphate formed on the cellulose during an acid hydrolysis extraction of NCC from a cellulose substrate such as wood.

The NCC may bear surface associated moieties which will bind a hydrophobic drug, for example the surfactant cetyl trimethylammonium bromide (CTAB) may ionically bind to the ionic groups on NCC and the bound CTAB will then bind the hydrophobic drug.

Numerous molecules could be synthesized to suit this purpose. Such molecules would contain some ionic groups to provide a charged interaction with NCC (preferably a positive charge to bind to negatively charged NCC) and a hydrophobic domain to bind hydrophobic drugs.

At high enough concentrations such CTAB molecules may form interlacing bilayers with hydrophobic cores but also with positively charged external faces. These systems may then bind both hydrophobic drugs in the hydrophobic core and ionic (charged) drugs on the outer face. One advantage of positively charged surfaces on NCC may be increased association with negatively charged mucous or tissue surfaces and increased local concentrations of drugs at preferred sites or even uptake of the entire NCC complex into the cell by endocytosis or pinocytosis mechanisms.

While reference has been made particularly to the use of the surfactant cetyl trimethylammonium bromide (CTAB) for ionically binding an ionic drug to NCC other binders may be employed, which have a hydrophobic domain for binding hydrophobic drugs, and ionic components, particularly a cationic charge, to ionically bind to anionic charges on the surface of NCC.

Further examples of molecules that might bind to NCC are amine or thiol conjugated diblock copolymers or amine or thiol conjugated hyperbranched polyglyerols. These molecules contain hydrophobic domains that may bind hydrophobic drugs. Such molecules would not be limited to ampipathic molecules since any hydrophobic polymer or molecule containing a hydrophobic domain could be used for such purposes. For example cationic amine groups are easily conjugated onto lactic acid and resulting polymerization reactions may give amine groups with hydrophobic poly lactic acid chains.

Thus within the invention, cationic moieties other than surfactants may be bound to the surface of the NCC to bind drugs. For example macromolecules such as the cationic polymer chitosan may bind to the surface and the excess positive charges may then bind negatively charged drugs such as antisense oligonucleotides or proteins. The macromolecule may form a coating and charged groups on the coating of macromolecules bind to the surface of NCC and oppositely charged drugs are bound to an outer surface of the coating. Although chitosan does not have a hydrophobic core there are many derivatives of chitosan that might include hydrophobic moieties.

The anionic sulphate groups on the surface of NCC may also be utilized to bind proteins. It is well known that the anionic sulphate ions may interact with cationic groups on proteins. See for example Levy DE et al (23) where immunoglobulins were shown to bind strongly to sulphated polysaccharides. Such binding methods might be used to deliver therapeutic proteins in a controlled manner especially as the binding interaction might stabilize the proteins. In certain situations antibodies or aptamers might be bound through sulphate interactions to allow for targeting/uptake of an NCC-drug complex to specific cells in the body.

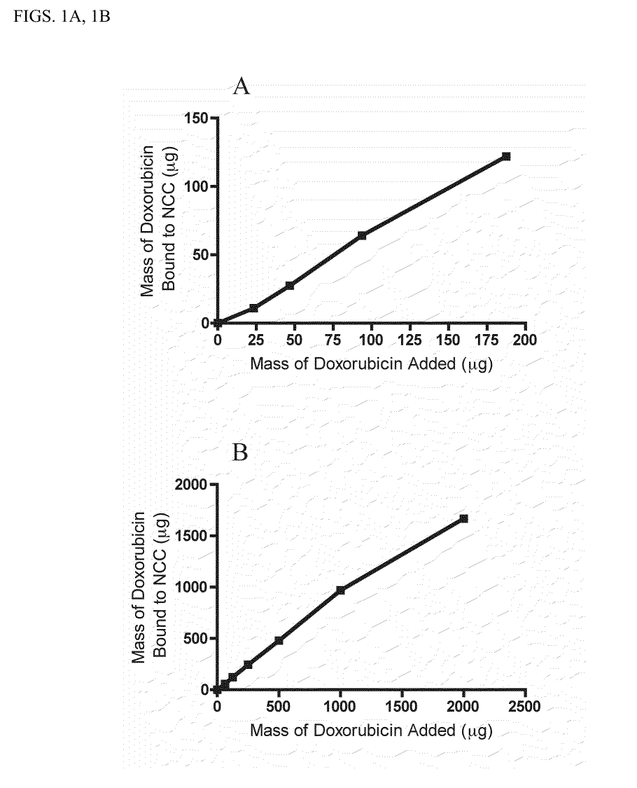

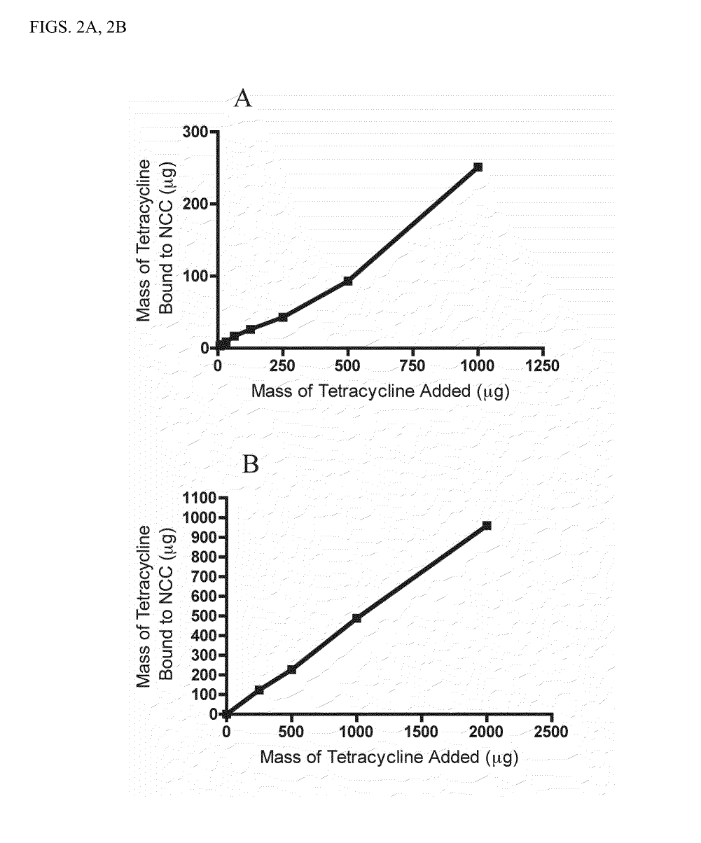

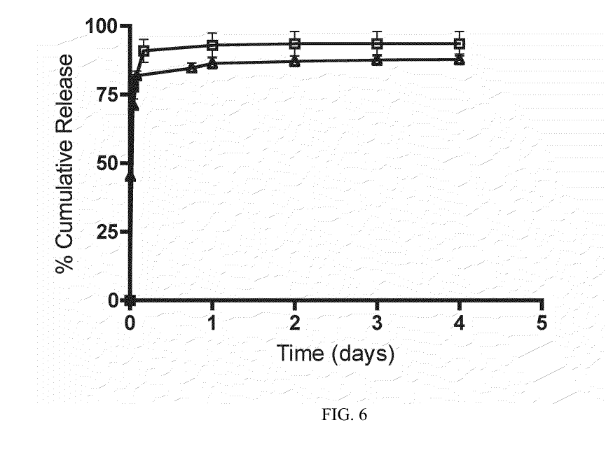

In accordance with one aspect of the invention hydrophilic drugs are bound directly to the surface of NCC at relatively high weight ratios (FIGS. 1 and 2) (e.g. almost 500 μg of tetracycline may be bound to just 2 mg of NCC, offering a 20% w/w drug loading—FIG. 2). The hydrophilic drugs such as tetracycline(TET) and doxorubicin (DOX) probably bind by an ionic interaction with the negatively charged surface of NCC since DOX is a cationic species slightly positively charged and TET is zwitterionic. Both these agents released rapidly from NCC in vitro (FIG. 6), probably due to PBS counterions displacing the drugs via ion exchange. This rapid release probably arose from interference with the NCC-drug ionic interaction by counter ions present in the PBS incubation media. Such rapid release profiles are also seen for acidic or basic drugs bound to ion exchange resins [14]. Nevertheless, these rapid release profiles observed for NCC may be suitable for potential applications as wound dressing materials or for implantation into surgical resection voids such as tumour removal sites or periodontal cavities.

In another aspect of the invention the NCC can be surface modified to deliver hydrophobic antiproliferative drugs. By coating the negatively charged NCC with a cationic surfactant such as cetyl trimethylammonium bromide (CTAB) it was possible to create a hydrophobic domain on the surface of the NCC.

In this latter aspect the hydrophobic drug is trapped or sequestered by the surfactant, the hydrocarbon chains of which may form micelles and admicelles on the surface of the NCC, and the hydrophobic drug is trapped between the adjacent hydrocarbon chains of the micelles, admicelles or both i.e. between a micelle and an adjacent admicelle pair.

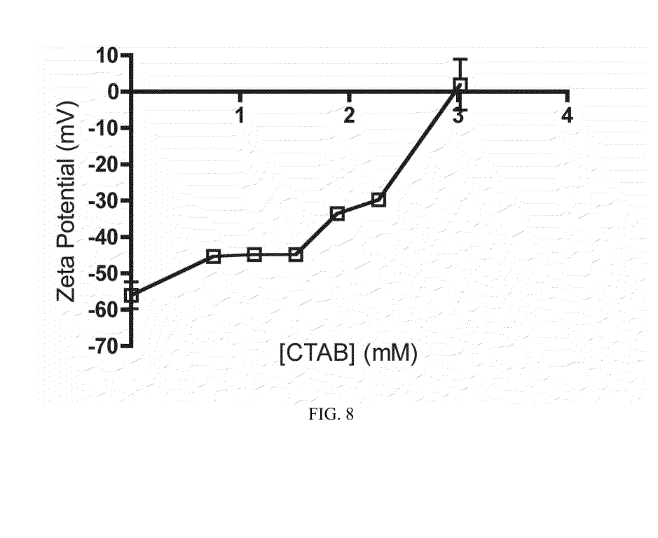

A clear interaction between CTAB and NCC was observed by flocculation phenomena at higher CTAB concentrations. Furthermore, the zeta potential (surface charge) on the NCC became increasingly less negative as the concentration of CTAB increased—evidence of a binding interaction between the CTAB and the NCC (FIG. 8). The strong association between NCC and CTAB was further supported by washing experiments whereby flocculation phenomena only began to decay when the NCC/CTAB nanocomplexes were washed more than 15 times with PBS (data not shown). An interaction between CTAB and negatively charged gold nanoparticles has been described whereby the surfactant binds to the surface of the particle and may cause concentration dependent particle aggregation [20]. Furthermore, Alkilany et al. [21] described the partitioning of hydrophobic napthol molecules into surfactant coated gold nanorods. Interestingly, although gold nanoparticles are used for laser induced thermal ablation of tumors, these particles have also been modified with hydrophobic polymer chains for the purpose of delivering hydrophobic drugs [22].

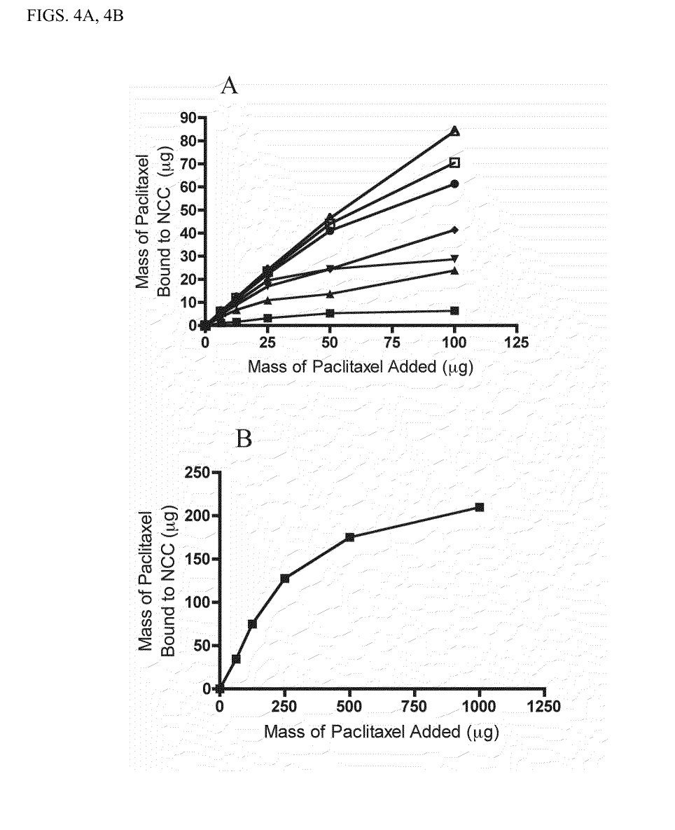

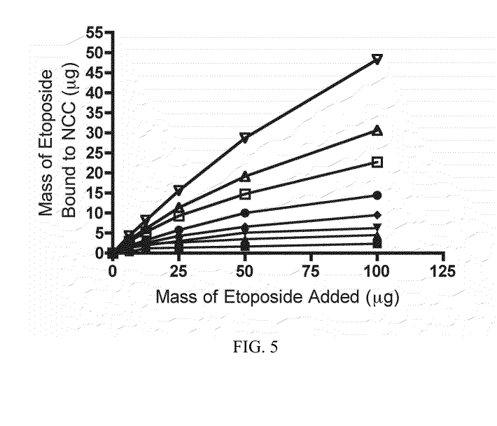

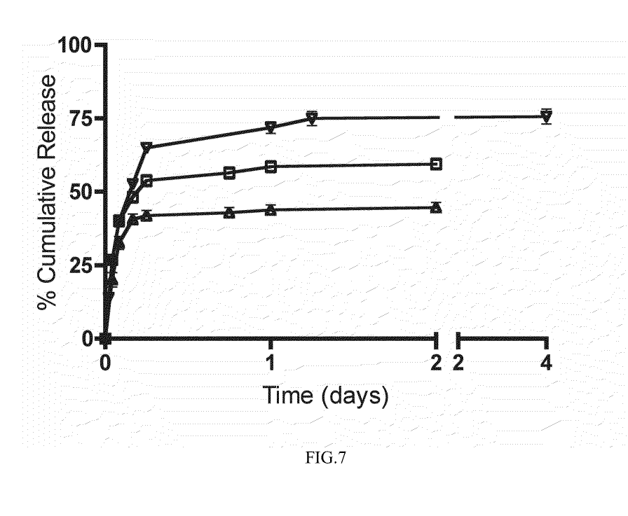

Hydrophobic drugs partitioned strongly into these CTAB domains on NCC using either free drug solutions at low concentrations or micellar solubilized drugs at higher concentrations (FIGS. 4 and 5). These drugs released more slowly from NCC (FIG. 7) than the hydrophilic drugs DOX and TET. However, the release profiles were all characterized by a burst phase of release of between 40% and 75% of the bound drug over the first 2 days followed by an extremely slow rate of release. These profiles suggest a weakly bound fraction of drug releasing quickly and a strongly bound fraction that released very slowly.

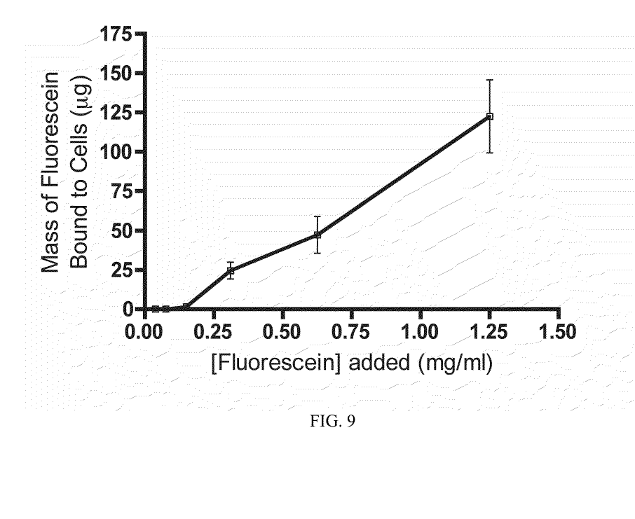

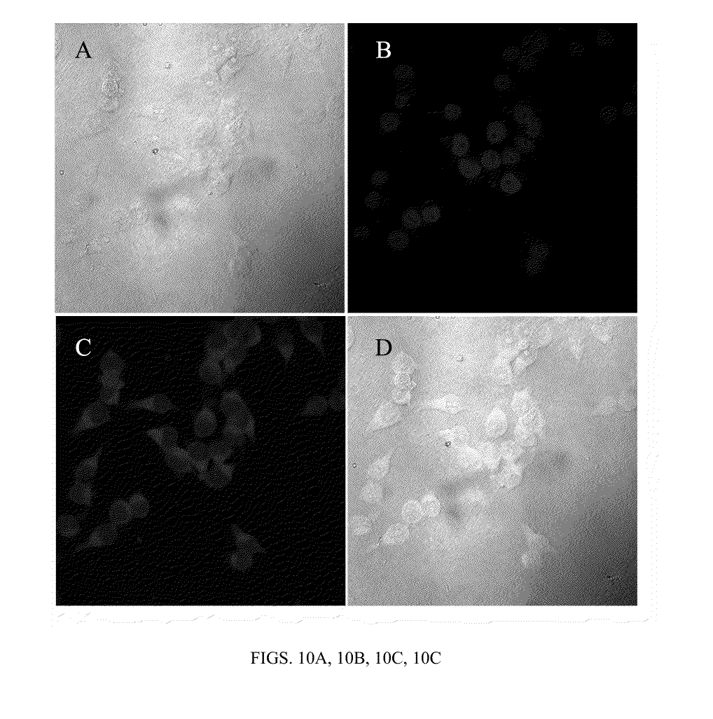

These NCC/CTAB nanocomplexes were shown to associate strongly with KU-7 cancer cells (FIG. 9). Because fluorescein was strongly bound within the CTAB coating on the NCC, it was possible to quantitate the cell-bound NCC by measuring the fluorescein emission from the cells. This assay does not differentiate between cell surface association and cellular uptake of NCC but clearly shows that NCC may be used to carry agents (in this case a hydrophobic probe, fluorescein) to cells. This concept is supported by confocal microscopy observations where a strong fluorescence signal from the cytoplasm of the cancer cells is indicated (FIG. 10). In these studies, the nuclear and cytoplasmic regions were differentially stained with DAPI (FIG. 10B) and fluorescein (FIG. 10C), respectively. No fluorescein signal was observed in the location of the nucleus, suggesting the cytoplasm as the location of fluorescein, since surface bound fluorescein would be observed over the full exposure of the cells. These data indicate cellular uptake of fluorescein but do not differentiate the uptake of free fluorescein from the NCC/CTAB/fluorescein nanosystem, as it is possible that fluorescein may partition into the hydrophobic cell membrane following cell binding of the nanosystem. Since cellular uptake of fluorescein was almost complete by 2 hours and anticancer drugs such as paclitaxel (PTX), docetaxel (DTX) and etoposide (ETOP) release occurred over days (FIG. 7), it may be assumed that NCC/CTAB/drug nanocomplexes offer a viable and novel method of delivering drugs to cells and may actually deliver these anticancer drugs as controlled release systems (NCC/CTAB/drug nanocomplexes) within cells. Confocal examinations further indicate good biocompatibility of the NCC-CTAB nanocomplexes, since cells were intact following incubation with the nanocomplexes for 24 hours. In cytotoxicity studies measuring the release of lactate dehydrogenase (LDH) (a marker of cytolysis), NCC and NCC-CTAB were found to have no lytic effect at a concentration of 1 mg/ml (data not shown). However, upon dilution in PBS, lower concentrations of NCC-CTAB (not NCC) were observed to cause some background lysis indicating that some unbound CTAB might interact directly with the cancer cell membranes.

Below are detailed procedures for NCC drug binding/release and evaluation for cell binding and uptake.

NCC Drug Binding Procedure: Doxorubicin hydrochloride (DOX) or tetracycline hydrochloride (TET), were dissolved in either 10 mM phosphate buffered saline (PBS) at pH 7.4, or dH₂O with increasing drug concentrations ([drug_{added}]). Drug solutions (1.5 ml) were added to 0.5 ml of NCC suspension in a 2 ml microcentrifuge tube and incubated at 37° C. with tumbling shaking at 8 rpm for 30 minutes. Suspensions containing PBS or NaCl produced flocculated NCC/drug suspensions, which were centrifuged at 18000×g for 10 minutes to pellet the NCC and bound drug. The concentration of unbound drug in the supernatant ([drug_{unbound}]) was assayed using a Varian 50 Bio UV Vis spectrophotometer (Varian, Inc., Mississauga, Ont.) using wavelengths of 482 nm and 364 nm for DOX and TET, respectively. The concentration of drug bound to the NCC ([drug_{bound}]) was calculated using the following equation:

[drug_{bound}]=[drug_{added}]−[drug_{unbound}] (1)

NCC does not flocculate in distilled water, therefore, the NCC/drug complexes prepared in distilled water could not be separated by microcentrifugation. In this case, the NCC/drug suspensions were transferred to dialysis bags with a molecular weight cut off of 10000 Da (Spectrum Laboratories, Inc., Rancho Dominguez, Calif.) and dialysed against distilled water overnight in the dark at 4° C. The concentration of unbound drug in the dialysate was determined by UV Vis spectroscopy, allowing for the calculation of the amount of drug bound to the NCC according to equation (1).

In order to solubilize the hydrophobic drugs paclitaxel (PTX), docetaxel (DTX) and etoposide (ETOP), the surface of the NCC was first modified with CTAB. This was achieved by incubating increasing amounts of CTAB with 2.5 mg of NCC so the final CTAB concentration varied from 0 mM to 12.9 mM. An aliquot of 100 mM NaCl was added, resulting in a final NaCl concentration of 10 mM, which facilitated flocculation and subsequent separation of the NCC/CTAB nanocomplexes by centrifugation as described above. The NCC/CTAB was incubated with stock solutions of the drugs with increasing concentrations. Since these drugs are characterized by low aqueous solubility, they were solubilized in a minimal amount of either DMSO or diblock copolymer in 10 mM NaCl as previously described [19]. The drug/NCC/CTAB suspensions were incubated at 25° C. with tumbling at 8 rpm for 30 minutes then centrifuged at 18000×g for 10 minutes to pellet the NCC/CTAB and bound drug. The amount of unbound drug in the supernatant was determined by HPLC using a Waters HPLC system with Millennium software and UV Vis detection. Separation was achieved using a Novapak™ C18 column with 20 μl injections and a mobile phase flow rate of 1 ml/min. The DTX and PTX mobile phase consisted of 58% acetonitrile, 37% dH₂O and 5% methanol with detection at 232 nm. The mobile phase for ETOP was 27% acetonitrile, 1% acetic acid and 72% dH₂O and detection was at 286 nm. Calibration curves were prepared for all drugs and were linear in the desired concentration range. The amount of drug bound to the NCC/CTAB was determined using equation (1).

Evaluation of Drug Binding to NCC: The amount of DOX bound to NCC increased significantly as the mass of drug added to the NCC suspension increased. When the dispersion medium was PBS, a maximum of 122 μg of DOX was bound to NCC representing a 65% binding efficiency (FIG. 1A). When distilled water was used as the dispersion medium, a maximum of 1667 μg of DOX was bound to NCC with a binding efficiency of 83% (FIG. 1B). It was found that the mass of TET bound to NCC was considerably less than that of DOX, regardless of the dispersion medium used (FIG. 2). Using PBS as a dispersion medium, a maximum of 251 μg of TET was bound with a 25% binding efficiency (FIG. 2A). When the dispersion medium was distilled water, 959 μg of TET was bound to NCC with a 48% binding efficiency (FIG. 2B).

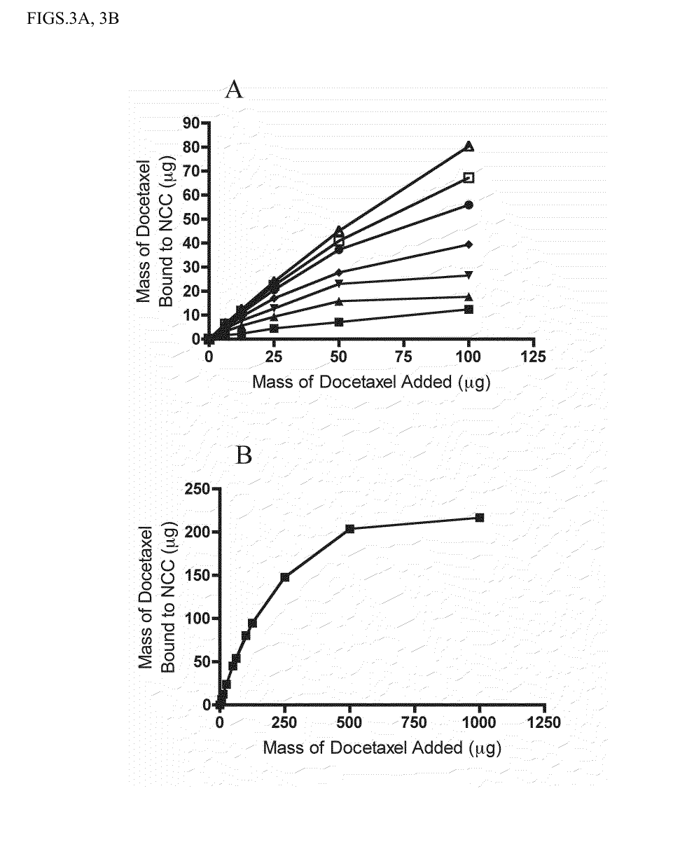

The effect of increasing concentration of CTAB coating on NCC on the binding of the hydrophobic drugs DTX, PTX and ETOP was investigated (FIGS. 3-5). In all cases it was found that increased amounts of CTAB resulted in increased drug binding. At the highest CTAB concentration (12.9 mM), the binding efficiency of DTX and PTX to the NCC/CTAB nanocomplexes was approximately 90% up to 100 μg of drug added (FIGS. 3A and 4A). Above this drug concentration, the drug binding efficiency decreased with saturation of binding occurring at approximately 200 μg (FIGS. 3B and 4B). Much less ETOP was capable of binding to the NCC/CTAB nanocomplexes with a 48% binding efficiency and a maximum of 48 μg bound when 100 μg of drug was added to the NCC/CTAB (FIG. 5).

Drug Release Procedure: DOX was bound to NCC for release studies by incubating a solution of 325 μg/ml of DOX in distilled water with a suspension containing 2.5 mg of NCC. In order to flocculate the NCC and allow for separation of the NCC/DOX nanocomplexes, NaCl was added to a final concentration of 10 mM. The suspension was centrifuged at 18000×g for 10 minutes to pellet the NCC/DOX and the drug binding was determined by UV Vis spectroscopy as described above. The final mass of DOX bound to the NCC for the release studies was 212±3.5 μg. The same procedure was used to prepare TET bound NCC nanocomplexes for the release studies, with the exception that the initial TET solution used was 1000 μg/ml, which resulted in the binding of 187±2.0 μg of TET. NCC/drug nanocomplexes with DTX, PTX and ETOP were prepared as described for the drug binding studies. The concentration of DTX and PTX that was incubated with the NCC suspension was 200 μg/ml and the concentration of ETOP was 100 μg/ml. The final mass of drug bound to the NCC was 184±4.8 μg, 149±4.8 μg and 63±0.1 μg for DTX, PTX and ETOP, respectively. The drug loaded NCC samples were resuspended in 1 ml of PBS followed by incubation at 37° C. with tumbling at 8 rpm. At predetermined times the suspensions were centrifuged at 18000×g for 10 minutes and the supernatant was removed for drug quantitation by UV Vis for DOX and TET or HPLC for DTX, PTX and ETOP, as previously described. At each sampling time point, fresh PBS was added to the tubes and the NCC/drug nanocomplexes were resuspended.

Evaluation of Release of Drugs from NCC: Both DOX and TET drug released rapidly from NCC resulting in approximately 90% of bound TET and 85% of bound DOX released in four hours (FIG. 8). By one day the drug release had plateaued with 93% and 87% of TET and DOX released, respectively. The release profiles of the hydrophobic drugs DTX, PTX and ETOP bound to NCC/CTAB are shown in FIG. 7. Approximately 26% of DTX was released within the first hour, followed by a slower more sustained release. In total, 59% of the total bound DTX was released in two days. A similar release profile was observed for PTX, which was characterized by a rapid release of 20% of bound drug in the first hour followed by slower release resulting in 44% drug release over two days. The release of ETOP was similar to DTX and PTX with the exception that a total of 75% of the drug was released over four days.

In distilled water NCC remained as a stable colloidal dispersion and did not flocculate or sediment under high-speed centrifugation. However, when 5 mM of NaCl was added, flocculation and subsequent sedimentation by high-speed centrifugation could be achieved. In water, CTAB had the same effect as NaCl so that at approximately 2 mM CTAB, the NCC could be sedimented under centrifugation. At lower concentrations of CTAB, a small amount of NaCl (10 mM) was added to tubes to enable sedimentation.

NCC had a strongly negative charge in water as evidenced by a zeta potential of approximately −55 mV. Upon incubation with CTAB, the zeta potential increased in a concentration dependent manner. At a concentration of 3 mM CTAB, there was complete neutralization of the negative zeta potential (FIG. 8).

Evaluation of Cell Binding and Uptake: More than 95% of the fluorescein bound to NCC/CTAB remained bound upon resuspension or dilution in aqueous media. When NCC/CTAB/fluorescein was incubated with KU-7 cells, the fluorescence signal was not quantifiable below 0.3 μg/ml; however, above this concentration, the fluorescence increased in a concentration dependent manner (FIG. 9). At concentrations greater than 1.25 mg/ml, there was no linearity of the fluorescein quantitation and it was not possible to accurately measure fluorescein uptake or binding to the cells.

The nuclei of the KU-7 cells displayed pronounced staining with DAPI as shown in FIG. 10B. There is clear evidence of cellular uptake of fluorescein as demonstrated by strong fluorescence emission from the cytoplasm of the cells (FIG. 10C). The uptake of fluorescein reached a maximum by two hours with little increase in cytoplasmic fluorescence emission after longer incubations. Cell uptake was observed using NCC/CTAB/fluorescein concentrations of 0.25, 0.5 and 1 mg/ml. There was no evidence of cell lysis with these complexes for up to 24 hours.

Pharmaceutical compositions of the invention may additionally contain a polymer and the polymer may contain one or more drugs other than that bound to the NCC.

### REFERENCES

[1] O'Connor R E and Schwartz J B 1993 Drug release mechanism from a microcrystalline cellulose pellet system Pharm Res 10 356-61
[2] Baumann M D, Kang C E, Stanwick J C, Wang Y, Kim H, Lapitsky Y and Shoichet M S 2009 An injectable drug delivery platform for sustained combination therapy J Control Release 138 205-13
[3] Bley O, Siepmann J and Bodmeier R 2009 Protection of moisture-sensitive drugs with aqueous polymer coatings: importance of coating and curing conditions Int J Pharm 378 59-65
[4] El-Maradny H A 2007 Modulation of a pulsatile release drug delivery system using different swellable/rupturable materials Drug Deliv 14 539-46
[5] Gomez-Carracedo A, Souto C, Martinez-Pacheco R, Concheiro A and Gomez-Amoza J L 2007 Microstructural and drug release properties of oven-dried and of slowly or fast frozen freeze-dried MCC-Carbopol pellets Eur J Pharm Sci 67 236-45
[6] Javadzadeh Y, Shariati H, Movahhed-Danesh E and Nokhodchi A 2009 Effect of some commercial grades of microcrystalline cellulose on flowability, compressibility, and dissolution profile of piroxicam liquisolid compacts Drug Dev Ind Pharm 35 243-51
[7] Khan G M and Zhu J B 1999 Studies on drug release kinetics from ibuprofen-carbomer hydrophilic matrix tablets: influence of co-excipients on release rate of the drug J Control Release 57 197-203

[8] Knight P E, Podczeck F and Newton J M 2009 The rheological properties of modified microcrystalline cellulose containing high levels of model drugs J Pharm Sci 98 2160-9
[9] Kranz H, Jurgens K, Pinier M and Siepmann J 2009 Drug release from MCC- and carrageenan-based pellets: experiment and theory Eur J Pharm Biopharm 73 302-9
[10] Podczeck F, Knight P E and Newton J M 2008 The evaluation of modified microcrystalline cellulose for the preparation of pellets with high drug loading by extrusion/spheronization Int J Pharm 350 145-54
[11] Watanabe Y, Mukai B, Kawamura K, Ishikawa T, Namiki M, Utoguchi N and Fujii M 2002 [Preparation and evaluation of press-coated aminophylline tablet using crystalline cellulose and polyethylene glycol in the outer shell for timed-release dosage forms] Yakuga Zasshi 122 157-62
[12] Abdalla A, Klein S and Mader K 2008 A new self-emulsifying drug delivery system (SEDDS) for poorly soluble drugs: characterization, dissolution, in vitro digestion and incorporation into solid pellets Eur J Pharm Sci 35 457-64
[13] Vinatier C, Gauthier O, Fatimi A, Merceron C, Masson M, Moreau A, Moreau F, Fellah B, Weiss P and Guicheux J 2009 An injectable cellulose-based hydrogel for the transfer of autologous nasal chondrocytes in articular cartilage defects Biotechnol Bioeng 102 1259-67
[14] Guo X, Chang R K and Hussain M A 2009 Ion-exchange resins as drug delivery carriers J Pharm Sci 98 3886-902
[15] Springate C M, Jackson J K, Gleave M E and Burt H M 2005 Efficacy of an intratumoral controlled release formulation of clusterin antisense oligonucleotide complexed with chitosan containing paclitaxel or docetaxel in prostate cancer xenograft models Cancer Chemother Pharmacol 56 239-47
[16] Springate C M, Jackson J K, Gleave M E and Burt H M 2008 Clusterin antisense complexed with chitosan for controlled intratumoral delivery Int J Pharm 350 53-64
[17] Shaikh S, Birdi A, Qutubuddin S, Lakatosh E and Baskaran H 2007 Controlled release in transdermal pressure sensitive adhesives using organosilicate nanocomposites Ann Biomed Eng 35 2130-7
[18] Lönnberg H, Fogelströma L, Samir M A S A, Berglunda L, Malmströma E and Hult A 2008 Surface grafting of microfibrillated cellulose with poly(ε-caprolactone)—Synthesis and characterization Eur Polym J 44 2991-7
[19] Zhang X, Jackson J K and Burt H M 1996 Development of amphiphilic diblock copolymers as micellar carriers of taxol Int J Pharm 132 195-206
[20] Yang Y, Matsubara S, Nogami M, Shi J and Huang W 2006 One-dimensional self-assembly of gold nanoparticles for tunable surface plasmon resonance properties Nanotechnology 17 2821
[21] Alkilany A, Frey R, Ferry J and Murphy C 2008 Gold nanorods as nanoadmicelles: 1-naphthol partitioning into a nanorod-bound surfactant bilayer Langmuir 24 10235-9
[22] Kim J-H and Lee T R 2006 Discrete thermally responsive hydrogel-coated gold nanoparticles for use as drug-delivery vehicles Drug Develop Res 67 61-9
[23] Levy D E, Horner A, Solomon A 1981 Immunoglobulin -sulphated polysaccharide interactions. J. Exp. Med. 153: 883-896.

1. A pharmaceutical composition comprising a drug bound to a carrier comprising nanocrystalline cellulose (NCC).

2. The pharmaceutical composition according to claim 1, wherein said drug is ionically bound to ionic groups on said NCC.

3. The pharmaceutical composition according to claim 1, wherein said NCC bears anionic acid groups which bind said drug.

4. The pharmaceutical composition according to claim 3, wherein said anionic acid groups which bind said drug, comprise sulphate groups.

5. The pharmaceutical composition according to claim 1, wherein said NCC is ionically bound to a surfactant; and said drug is bound by the surfactant to said NCC.

6. The pharmaceutical composition of claim 1, wherein charged groups on adsorbed molecules containing hydrophobic groups are bound to the surface of NCC to provide a hydrophobic moiety on the NCC so that hydrophobic drugs may be bound to the hydrophobic moiety.

7. The pharmaceutical composition of claim 6, where the adsorbed molecules are selected from amine or thiol terminated hydrophobic polymers, amine or thiol terminated diblock or triblock copolymers, dendrimers and hyperbranched copolymers.

8. The pharmaceutical composition of claim 7, where the adsorbed molecules are selected from poly lactic acid and polycaprolactone.

9. The pharmaceutical composition of any one of claims 6 to 8, wherein the drug is bound within the hydrophobic section of the surface bound molecules.

10. The pharmaceutical composition of claim 1, wherein charged groups on a coating of macromolecules bind to the surface of NCC and oppositely charged drugs are bound to an outer surface of the coating.

11. The pharmaceutical composition of claim 10, wherein the coating of macromolecules comprises chitosan.

12. The pharmaceutical composition of claim 1, wherein anionic sulphate groups on said NCC bind proteins for therapeutic protein delivery or targeting protein- or apatamer-cell binding.

13. The pharmaceutical composition according to claim 5, wherein said drug is trapped or sequestered by micelles or admicelles of the surfactant and/or bound via charge interactions to the outer surface of positively charged admicelles.

14. The pharmaceutical composition of any one of claims 1 to 13, further comprising a polymeric material.

15. The pharmaceutical composition of claim 14, where the polymeric material contains another drug or drugs other than that bound to the NCC.

16. A process of producing a pharmaceutical composition comprising binding a drug to a carrier, said carrier comprising nanocrystalline cellulose (NCC).

17. The process according to claim 16, wherein said binding comprises ionically binding said drug to ionic groups on said NCC.

18. The process according to claim 16, wherein said binding comprises ionically binding a surfactant to ionic groups on said NCC, and binding said drug with said surfactant.

19. The process according to any one of claims 16 to 18, further comprising forming said composition into a dosage form.

20. The process according to claim 19, wherein said dosage form is a tablet.

21. In a method of treating or preventing a disease or ailment in which a drug is administered in a dosage form to a patient in need, the improvement wherein said drug is bound to a carrier comprising nanocrystalline cellulose (NCC).

22. Nanocrystalline cellulose (NCC) for use as a carrier for a drug in a pharmaceutical composition.

23. The nanocrystalline cellulose (NCC) according to claim 22, wherein the drug is bound to the NCC.

24. The nanocrystalline cellulose (NCC) according to claim 23, wherein the drug is ionically bound to the NCC.

25. The nanocrystalline cellulose (NCC) according to claim 22, wherein a surfactant is ionically bound to the NCC and the drug is bound by the surfactant.

26. Use of nanocrystalline cellulose (NCC) in the manufacture of a pharmaceutical composition in which the NCC is a carrier for a drug.

27. The use of claim 26, wherein the pharmaceutical composition contains polymeric materials.

28. The use of claim 27, where the polymer may contain another drug or drugs other than that bound to the NCC.
